(19) United States
(12) Patent Application Publication
(10) Pub. No.: US 2022/0087622 A1
(43) Pub. Date: Mar. 24, 2022

(54) MULTIMODAL IMAGING SYSTEM AND METHOD

(71) Applicant: MILABS B.V., UTRECHT (NL)

(72) Inventor: Frederik Johannes BEEKMAN, Utrecht (NL)

(73) Assignee: MILABS B.V., UTRECHT (NL)

(21) Appl. No.: 17/423,283

(22) PCT Filed: Jan. 17, 2020
(86) PCT No.: PCT/NL2020/050025

§ 371 Date: Jul. 15, 2021

(30) Foreign Application Priority Data
Jan. 17, 2019 (NL) 2022409

(51) Int. Cl. A61B 6/03; A61B 6/06; A61B 6/00; A61B 6/04

(52) U.S. Cl. CPC A61B 6/037; A61B 6/032; A61B 6/54; A61B 6/4411; A61B 6/0487; A61B 6/06

(57) ABSTRACT

An imaging system for obtaining images of a patient includes a first scanning device, for example a CT scanner or an MRI scanner, and a second scanning device, being a SPECT scanner device. A mover mechanism moves a patient carrier in a first direction into the first imaging space of the first scanning device. The SPECT scanner device is positioned behind the first scanning device. The mover mechanism moves the patient carrier further in the first direction into the SPECT imaging space. The SPECT scanner device includes a housing accommodating a set of detectors. The imaging system further includes a plurality of exchangeable collimator assemblies with a first and a second collimator assembly, the assemblies having different imaging properties. The SPECT scanner device is configured for removal and introduction of each one of said plurality of exchangeable collimator assemblies at a side of the housing thereof that faces away from the first scanning device. A collimator support and conveyance system is configured to support and convey each collimator assembly in replacing one of the plurality of exchangeable collimator assemblies that is positioned in the SPECT scanner around the SPECT imaging space for another one of the plurality of exchangeable collimator assemblies.

### FIELD OF THE INVENTION

The present invention relates to an imaging system and method for obtaining images of a patient. The imaging system comprises a first scanning device, not being a SPECT scanner device, which first scanning device has a first imaging space and is configured to obtain scanning images of at least a part of the patient. For example, the first scanning device is embodied as a CT scanner or an MRI scanner. The imaging system also comprises a second scanning device, being a SPECT scanner device, which second scanning device has a SPECT imaging space and is configured to obtain SPECT images of at least a part of the patient.

Such a combined scanning imaging system serves to examine the patient for certain medical conditions or other properties. Herein, use is made of nuclear radiation and it is desirable to limit the radiative load of the patient as much as possible.

### BACKGROUND OF THE INVENTION

Presently known combined, i.e. multimodal, scanning systems often provide a less than optimal combination of scanning speed, sensitivity, and operational flexibility. Known multimodal imaging systems are considered to require a less desirable radiative load on the patient.

### OBJECT OF THE INVENTION

It is an object of the present invention to provide an improved multimodal imaging system, e.g. an imaging system that allows for enhanced versatility and/or enhanced sensitivity and/or enhanced operational flexibility, e.g. allowing for a reduction of radiative dose for scanning a patient.

### SUMMARY OF THE INVENTION

The invention provides for an imaging system.

The imaging system comprises:
a first scanning device, not being a SPECT scanner device, which first scanning device has a first imaging space and is configured to obtain scanning images of at least a part of the patient, the first scanning device for example being embodied as a CT scanner or an MRI scanner, and
a second scanning device, being a SPECT scanner device, which second scanning device has a SPECT imaging space and is configured to obtain SPECT images of at least a part of the patient.

The imaging system comprises a patient carrier adapted to carry the patient, for example a patient table that is configured to support the patient lying on the patient table. A mover mechanism, e.g. a table mover mechanism, is configured to move the patient carrier in a first direction A allowing to introduce at least a part of the patient carried by the patient carrier into the first imaging space of the first scanning device, e.g. from an initial patient receiving position outside the first scanning device in front of the first scanning device.

The SPECT scanner device is positioned behind the first scanning device when seen in the first direction A.

The mover mechanism is configured to move the patient carrier further in the first direction A allowing to introduce at least a part of the patient carried by the patient carrier into the SPECT imaging space of the SPECT scanner device.

The SPECT scanner device comprises a housing accommodating a set of detectors in an arrangement around the SPECT imaging space, for example the set of detectors being stationary during obtaining of the SPECT images.

The imaging system further comprises a plurality of exchangeable collimator assemblies.

The SPECT scanner device is configured to receive one of said plurality of exchangeable collimator assemblies in a position around the SPECT imaging space, allowing to obtain SPECT images of at least a part of the patient using one or more of the detectors of the set of detectors.

The plurality of exchangeable collimator assemblies comprises at least a first collimator assembly and a second collimator assembly having different imaging properties.

The SPECT scanner device is configured for removal and introduction of each one of said plurality of exchangeable collimator assemblies at a side of the housing thereof that faces away from the first scanning device, e.g. at the rear side of the SPECT scanner device with the front side of the SPECT scanner device towards the rear side of the first scanner device. Preferably this removal and introduction is done in said first direction and counter to said first direction respectively.

The imaging system further comprises a collimator support and conveyance system that is configured to support and convey each collimator assembly of the plurality of exchangeable collimator assemblies in replacing one of the plurality of exchangeable collimator assemblies that is positioned in the SPECT scanner around the SPECT imaging space for another one of the plurality of exchangeable collimator assemblies.

In practical embodiments, each exchangeable collimator assembly associated with the SPECT scanner device comprises not only one or more collimator components that make up the collimator but also one or more shielding components that form an annular axial shield that extends about one of the axial openings of the imaging space of the collimator, e.g. said annular shield fitting into a corresponding opening in the housing of the SPECT scanner device, e.g. an opening in a shielding component of the housing of the SPECT scanner.

The imaging system may be provided with a SPECT scanner of a type with optimum detector use, and sensitivity. Due to the arrangement of the SPECT scanner device in series, seen in the first direction, behind the first scanner device, there will be no interference between the scanner devices.

Operational flexibility for the SPECT scanner device is achieved by having exchangeable collimator assemblies that are introduced and removed at the rear, so from behind, i.e. from the opposite side as where the first scanning device is positioned.

In a preferred structural design, the imaging system is configured such that the imaging system allows for replacing one of the plurality of exchangeable collimator assemblies that is positioned in the SPECT scanner around the SPECT imaging space for another one of the plurality of exchangeable collimator assemblies without affecting operability of the first scanner device. For example, in an embodiment, it is possible for a SPECT collimator assembly to be replaced while the patient is positioned by the patient carrier in a position that allows for scanning of the patient with the first scanning device, i.e. without having to displace the patient and/or the patient carrier for the purpose of exchanging the collimator assemblies.

In an embodiment, the optimum collimator assembly for SPECT scanning the patient is selected even while obtaining scanning images with the first scanning device. This in turn allows to limit the radiative load on the patient, as well as to limit the time needed for the total scanning procedure.

In embodiments, the first scanning device in particular comprises a CT scanner or an MRI scanner. These types of scanning devices are useful scanning devices in and by themselves, and also are able to provide a map of the patient's body, on which to register the scanning images taken by the SPECT scanner. In addition, the first scanning device in general, i.e. of any appropriate kind, may serve to perform a first (type of) scanning of the patient's body, so that the operator, or a suitably embodied control unit processing the scanning images of the first scanning device, may subsequently select one of the exchangeable collimators for further scanning with the SPECT scanner.

In this application, a CT scanner is understood to be X-ray computed tomography imaging apparatus.

In practical embodiments, the mover mechanism is configured to move the patient carrier, e.g. a patient table, into an initial patient receiving position outside of said first scanning device, e.g. in front of the first scanning device, along the first direction into a position wherein at least a part of the patient is within the imaging space of the first scanning device, and further in the first direction into a position wherein a part of the patient is within the imaging space of the SPECT scanner. It goes without saying that the mover mechanism is configured to move the patient vice versa, i.e. out of the SPECT scanner and from there out of the first scanner device.

For example, the mover mechanism has a base configured to stand on a floor, e.g. at a fixed location or a wheeled embodiment or the like, in front of the first scanner device, wherein the patient table is mobile, in practice by an actuator arrangement of the mover mechanism, relative to the base allowing to move the patient from an initial position outside the first scanner device into the imaging space of the first scanner device in the first direction and, when desired, further in the first direction into the imaging space of the SPECT scanner device.

Note that the collimator carrying system may comprise several separate carrying devices, such as one for each (or every two) collimators, as long as each collimator may be positioned in the SPECT scanner individually.

Advantageous embodiments are described in the dependent claims as well as in the now following part of the description.

In embodiments, at least the one of the plurality of exchangeable collimators is a full-ring collimator, configured to extend substantially completely around the SPECT imaging space during obtaining of the SPECT images. Herein, “full-ring” indicates that the collimator extends around the imaging space, and as a whole forms a closed unit, although gaps in the sensitive detecting surface offered by the set of detectors may be present in embodiments. Such a full-ring collimator, together with the set of detectors around it, allows optimum sensitivity, which again is advantageous for a low radiative load and for patient well-being.

In embodiments, neither one or more of the exchangeable collimator assemblies and/or the set of detectors, needs to have a circular “ring”-shape. It is also well possible that one or more of the exchangeable collimator assemblies and/or the set of detectors has/have a polygonal cross-section, such as a triangle, a pentaeder, a hexaeder and so on.

As an alternative to the full-ring collimator assembly, it is also possible to have other types of collimator cross-sectional shapes, such as a “partial-ring” collimator that extends about a part, e.g. about two thirds, of a full circle. The latter allow more targeted scanning of smaller regions of interest, for e.g. cardiac or breast testing. For such collimators, the set of detectors need not extend around the SPECT imaging space completely. Yet, it is desirable for the set of detectors to extend around the imaging space completely in order to be as versatile as possible.

In embodiments, the imaging system comprises a collimator rotator for rotating at least one of the plurality of exchangeable collimators around the SPECT imaging space over at least a predetermined angle. This allows to obtain more angular information, while still enjoying the high sensitivity. Note that it suffices to rotate the collimator proper, while the shielding and detector device may remain static. The predetermined angle may be as small as the repetition angle of a polygonal cross-section collimator, or half that angle when wishing to just double the sampling. For example, in case of a hexaeder cross-section, a 60 degree rotation angle (or 30 degrees in case of double sampling only) may suffice. In such non 360 degree cases, the collimator may also be rotated back and forth over such angle.

In embodiments, the mover mechanism and/or the patient carrier are arranged to move the patient carrier in at least one direction perpendicular to the first direction, and preferably in two mutually perpendicular directions that are perpendicular to the first direction, i.e. the first direction being in horizontal X direction, the mover mechanism also providing for motion in horizontal Y direction and/or vertical Z direction, and/or any tilting of the carrier about one or more tilt axes. This allows enhanced flexibility in positioning the patient or a region of interest in the patient, with respect to the first and/or second scanner device.

In embodiments, at least one, and in particular each, of the plurality of collimator assemblies is one of a focused pinhole collimator, cross-slit pinhole collimator or slit-slat pinhole collimator.

Herein, a focused pinhole/cross-slit/slit-slat collimator has pinholes or effective pinholes with overlapping fields of view that determine a, relatively small, focus volume. For cross-slit collimators, reference is made to e.g. WO07133068A2, and for slit-slat collimators, reference is made to document WO15152720A2, which documents are incorporated herein by reference in their entirety for all purposes.

As is known per se, full-ring focused pinhole collimators provide a better sensitivity than conventional parallel collimators, that either have relatively much unused detector surface, or a relatively large distance to the target region or suffer from strong resolution degrading effects. Yet, it is not excluded that the plurality of collimators comprises one or more of these parallel hole collimators or other types of collimators.

In embodiments, it is envisaged that an operator manually exchanges the collimator assembly in the SPECT scanner, for example the collimator support and conveyance system comprising a manually movable cart on which the assembly is supported, e.g. a wheeled cart. The cart may also be provided with a drive motor and/or with a steering arrangement, e.g. independently driven wheels allowing to steer the cart. As explained herein the collimator assembly is heavy.

In embodiments, it is envisaged that the replacing of one collimator assembly for another one in the SPECT scanner device is done by means of an automated system.

In embodiments, the collimator support and conveyance system is an automated collimator support and conveyance system that is configured to automatically exchange a collimator assembly that is located in an operable position thereof where the collimator assembly surrounds the SPECT imaging space for another one of the plurality of exchangeable collimator assemblies, which other one collimator assembly is in a storage position thereof prior to said automated exchange.

In embodiments, the collimator support and conveyance system comprises a movable cart or a suspended carrier, in particular one cart or suspended carrier for each collimator. A collimator cart is a useful tool to carry and replace the collimator assemblies, that helps in positioning the collimator assembly with respect to the SPECT scanner.

The collimator assemblies may be provided with an identification device, such as an RFID tag or the like, for automatic recognition in the SPECT scanner device, so that the SPECT scanner may be controlled on the basis of the recognized collimator assembly, such as for adapting settings thereof.

In embodiments, at least two of the plurality of collimators, in particular the first collimator and the second collimator, are provided on a common carrier, the collimator replacer comprising a carrier rotator arranged to at least rotate the common carrier around 180° in a horizontal plane. It is pointed out that such a collimator carrying cart is e.g. known from document US2013082450A1, which document is deemed incorporated by reference for all purposes.

In embodiments, the imaging system comprises a control unit arranged to control the first scanner device and the SPECT scanner device, wherein the control unit is further arranged to process the obtained scanning images of the first scanner device and to provide control instructions to at least one of the SPECT scanner device and the mover mechanism based on the processing of the obtained images of the first scanning device. Such controlling may comprise the registering of the SPECT image onto the first scanning images, such as CT or MRI images and selecting the area of the body to which SPECT scanning is applied. Since the patient's position on the table is also known during moving of the table, the patient, or a relevant part thereof, is easily positioned optimally with respect to the SPECT scanner. The control unit may be arranged to process the obtained images during scanning with the first scanning device. This enables a quick and efficient second step of scanning in the SPECT scanner, which is helpful for patient well-being.

In embodiments, the processing comprises determining a region of interest in the patient based on the obtained scanning images of the first scanning device, and the control instructions comprise instructions to move the mover mechanism and to control the SPECT scanner such as to obtain one or more SPECT images of said determined region of interest. The obtained first scanning images may be processed automatically by the control unit. Of course, operator input or monitoring is also possible. Based on the first scanning images, one or more regions of interest (ROIs) are determined, either by an operator or automatically by the control unit, such as based on the measured intensity distribution or other predetermined criteria. The control unit then determines how to position the patient with respect to the SPECT scanner by means of the mover mechanism.

In embodiments, the control instructions comprise a selection instruction to select one collimator assembly from the plurality of collimator assemblies, based on the processing of the obtained scanning images of the first scanning device. In embodiments, the control unit is arranged to control the collimator support and conveyance system to automatically position the selected collimator assembly around the SPECT imaging space, based on the selection instruction. In these embodiments, the control unit may, either autonomously and/or with operator input, determine the best available collimator assembly for further scanning of the patient. This collimator assembly may be positioned by an operator, or by the collimator support and conveyance system, preferably automatically, and under the control of the control unit. In addition the control unit may provide optimum positioning of the patient with respect to that best available collimator assembly. This is advantageous in terms of sensitivity and scanning time, which is, again, beneficial for the patient. The control unit is then arranged to determine such best available collimator shortly after, or possibly even during, scanning in the first scanning device, so that the shortest possible total scanning time may be achieved.

The invention also relates to a method for exchanging collimator assemblies in an imaging system as disclosed herein.

The invention also relates to a method for exchanging collimator assemblies in an imaging system as disclosed herein, wherein the collimator support and conveyance system is used in removing one of the plurality of exchangeable collimator assemblies that is positioned in the SPECT scanner device around the SPECT imaging space at the side of the housing of the SPECT scanner device that faces away from the first scanning device and is used in introduction of another one of the plurality of exchangeable collimator assemblies at the side of the housing of the SPECT scanner device that faces away from the first scanning device, preferably said removal and introduction in said first direction and counter to said first direction respectively.

It is envisaged as an embodiment that the removing of one of the plurality of exchangeable collimator assemblies and introduction of another one of the plurality of exchangeable collimator assemblies at the side of the housing of the SPECT scanner device that faces away from the first scanning device is done whilst a patient is carried by the patient carrier is in a position within the first imaging space of the first scanner device, e.g. whilst the patient is being scanned using the first scanner device. This may enhance efficient use of the system and/or shorten the time required for imaging a patient which may bring along a reduction of the radiative load.

In an embodiment, the imaging system further comprises a control unit that is linked to each of the first scanner device and the SPECT scanner device, which control unit is configured to control each of the first scanner device and the SPECT scanner device, wherein the control unit is further configured to provide a selection instruction to select one collimator assembly from the plurality of exchangeable collimator assemblies, based on a processing of the obtained scanning images of the first scanning device, e.g. said selection instruction being displayed for use by an operator of the imaging system, e.g. said operator then selecting a cart supporting the selected collimator assembly.

In an embodiment, the control unit is configured to supply the control instruction to an automated collimator support and conveyance system in order to automatically position the selected collimator assembly around the SPECT imaging space.

The invention will now be explained further with reference to the drawings.

### BRIEF DESCRIPTION OF THE DRAWINGS

In the drawings:

FIG. 1 diagrammatically shows an example of an imaging system for imaging a patient according to the present invention,

FIG. 2 shows an example of an exchangeable collimator assembly and a cart for in an imaging system according to the present invention,

FIG. 3 illustrates a SPECT scanner device having a housing with an opening at the side facing way from the first scanner device of the imaging system of the present invention, wherein the exchangeable collimator assembly support by the cart of FIG. 2 is in position surrounding the imaging space of the SPECT scanner,

FIG. 4 illustrates another example of an imaging system for imaging a patient according to the present invention.

### DETAILED DESCRIPTION OF EMBODIMENTS

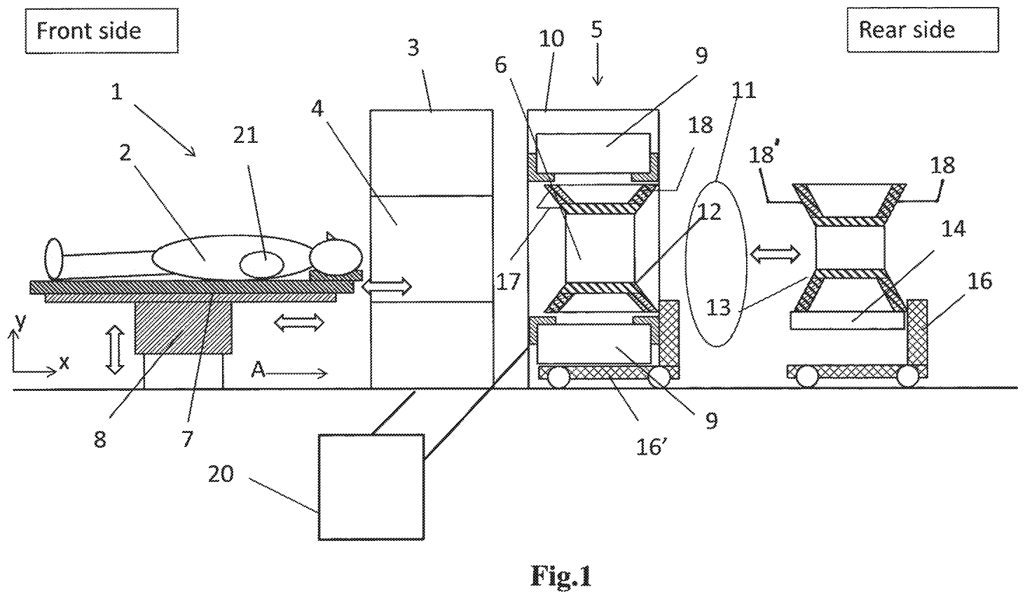

The FIG. 1 diagrammatically shows an example of an imaging system 1 according to the present invention for imaging a human patient 2.

In this example the imaging system 1 comprises a first scanner device 3, here embodied as a CT scanner 3 with a CT imaging space 4, and a second scanner device 5 embodied as a SPECT scanner 5 with a SPECT imaging space 6.

For scanning the patient 2 with the system 1, the patient lies on table 7.

The table 7, with the patient 2 lying thereon, is movable by means of a mover mechanism 8 associated with the table 7.

It is illustrated that the mover mechanism has a base configured to stand on a floor, e.g. at a fixed location or a wheeled embodiment or the like, in front of the first scanner device 3. The patient table 7 is mobile, in practice by an actuator arrangement of the mover mechanism 8, relative to the base allowing to move the patient from an initial position outside the first scanner device into the imaging space of the first scanner device in a first or X-direction (arrow A) and, when desired, further in the first direction A into the imaging space of the SPECT scanner device 5.

The SPECT scanner 5 comprises a set of detectors 9 accommodated in a housing 10 of the scanner 5.

An exemplary region of interest in the patient 2 is indicated with reference numeral 21.

The first scanner of the imaging system 1 is in this case a CT scanner 3, or more generally an X-ray tomography device. Alternatively, it may for example be “C-arm” device or an MRI scanner.

The CT scanner 3 has a CT scanning space 4 for accommodating a patient 2 on a table 7.

The table 7 can be moved in X-direction (arrow A) by means the mover mechanism 8.

The table 7 with the patient 2 is moved into the CT scanning space 4 from the side facing away from the SPECT scanner 5, i.e. in the FIG. 1 from the left side, that is the front side of the device 3, in the direction of the arrow indicated A.

Here, the table 7 is moved by means of the table mover mechanism 8, which allows movement in the “A”-direction, corresponding to the positive x-direction in the FIG. 1, as well as optionally in the y-direction and/or the (perpendicular) z-direction for optimum positioning of the patient relative to the scanner devices.

During scanning with the CT scanner 3, scanning images are obtained, that are processed by the operatively connected control unit 20, e.g. by an image processing program run on a computer of the control unit 20. In embodiments, based on these obtained CT scanning images, and/or based on knowledge beforehand, the control unit 20 is able to determine a region of interest 21 in the patient, and its position in space, i.e. on the table 7. For example, the CT scanner 3 obtains scanned images from which the control unit 20 generates a 3D image of the patient 2. Based on knowledge of which body part of the patient 2 is of interest, the control unit 20 is then able to determine where this body part, i.e. the region of interest 21, is present on the table 7.

The control unit 20 may be embodied to check, based on the obtained CT images, whether a collimator assembly that is at said moment already present in the SPECT scanner 5 is the best available collimator assembly in a set 11 of exchangeable collimator assemblies 12, 13 of the system in view of selection of the optimal collimator assembly

For example, such a selection of the optimal collimator assembly may be based on the dimensions and/or position and/or depth of the region of interest 21 in the patient 2, as determined by means of the first scanner device 3, and/or be based on a required sensitivity and/or resolution for scanning the region of interest 21.

In the FIG. 1, it is illustrated that a first collimator assembly 12 is present in the SPECT scanner device 5. As explained, this collimator assembly 12 may not be the best available collimator assembly of the set of assemblies 12, 13 of the system 1. This determination may be made solely by, or assisted by, the scanning of the patient with the first scanner device 3, e.g. based on a computerized processing of images obtained by the first scanner device 3. If the assembly 12 is not the best available collimator it is envisaged that the assembly 12 is replaced by a best suited collimator assembly of the system 1, here the second collimator assembly 13.

It is illustrated in FIG. 1 that each of the collimator assemblies 12, 13 is mounted on a respective movable cart 16′, 16. Each cart 16, 16′ forms a component of a collimator support and conveyance system that belongs to the system 1 and is used in replacing one of the plurality of exchangeable collimator assemblies that is positioned in the SPECT scanner around the SPECT imaging space for another one of the plurality of exchangeable collimator assemblies, here for example assembly 12 being replaced by assembly 13. As will be appreciated a SPECT collimator assembly adapted for use in a SPECT scanner that can receive in the imaging space thereof a human patient lying on a table has significant dimensions and thus a corresponding very significant weight.

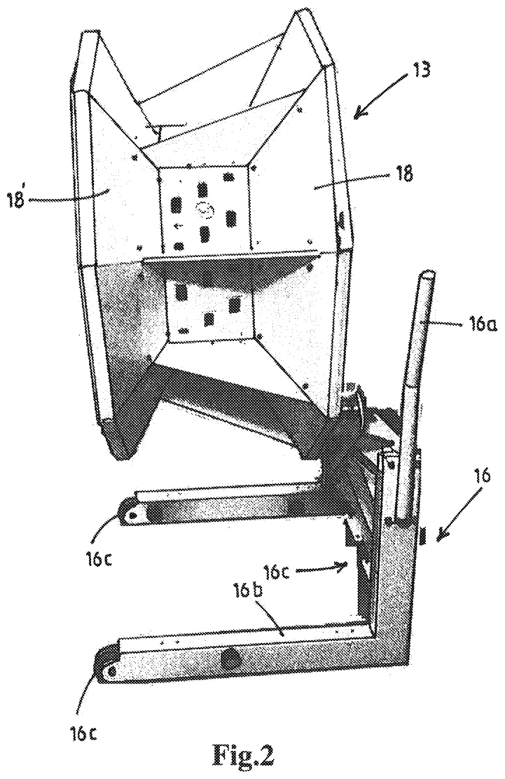

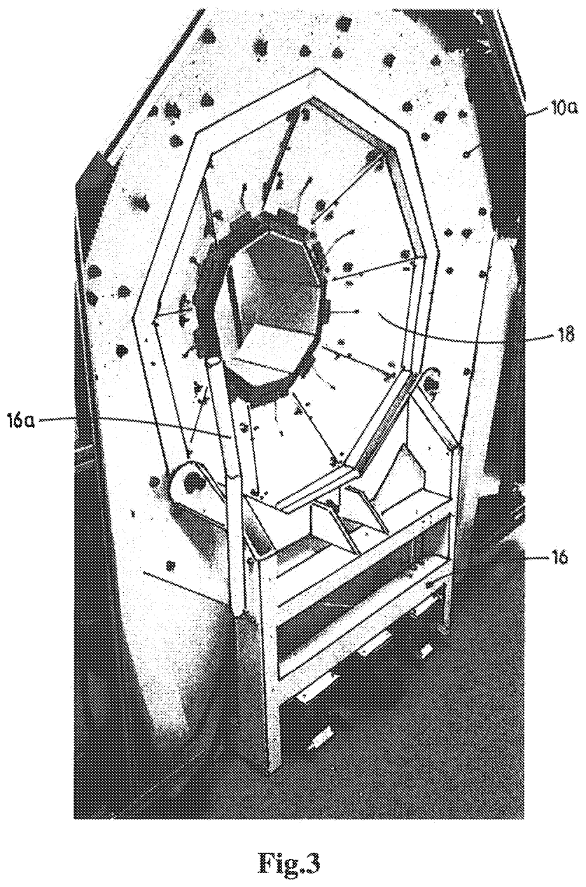

Each cart 16, 16′ may be movable by hand, e.g. the cart having a handle 16a as shown in FIGS. 2, 3. In another embodiment a cart 16, 16′ is movable by means of a non-shown cart mover/motor, e.g. mounted on the cart itself.

A motor of a motorized cart 16, 16′ supporting one or more collimator assemblies 12, 13 may be linked to the control unit 20 as part of an automated collimator support and conveyance system that is configured to automatically exchange a collimator assembly that is located in an operable position thereof where the collimator assembly surrounds the SPECT imaging space for another one of the plurality of exchangeable collimator assemblies, which other one collimator assembly is in a storage position thereof prior to said automated exchange.

In embodiments, as shown in FIGS. 1, 2, 3, and 4, a chassis 16b, e.g. provided with wheels 16c, of the cart 16, 16′ is moved to under the region of the detectors 9 of the SPECT scanner 5 as this enhances stability of the cart 16, 16′ supporting the heavy assembly 12, 13.

Alternatively, it is also possible to provide the two collimators 12 and 13 on a single cart, which may then be provided with a rotator device, such that the desired collimator 12, 13 may be inserted into the SPECT scanner 5.

In general, it is also possible to provide in the set of exchangeable collimators for the SPECT scanner 5 more than two collimators, such as three, four, . . . collimators, and so on. The collimator support and conveyance system will then be correspondingly adapted, such as one cart for two collimators or the like. It is also possible to use a different collimator support and conveyance system, such as one wherein the collimator assemblies are suspended from an overhead rail system, e.g. the rail system being mounted to a ceiling with a rail system, a frame on legs from which the collimator assemblies hang, or the like.

It is illustrated in FIG. 1 that the first collimator assembly 12 supported by the cart 16′ is replaced by the second collimator assembly 13 support by the cart 16, wherein the second collimator assembly has different imaging properties, e.g. better suited for the determined region of interest 21 in the patient 2. As is illustrated this replacement is done at the rear side of the SPECT scanner, that is the side of the SPECT scanner 5 which faces away from the CT scanner 3.

As explained it is envisaged that the replacement of the one collimator assembly 12 in the SPECT scanner device 5 for a more suited collimator assembly 13 can be performed, or at least initiated, whilst the patient 2 lies on the table 7, e.g. whilst the patient 2 is being scanned using the first scanner device 1. So the table 7 and the patient 2 thereon, whilst being scanner by scanner 3 do not interfere with replacing of the SPECT collimator. In fact, the CT scanning may even continue while the SPECT collimator assembly is being replaced.

For example, the collimator assemblies 12, 13 each comprise a full-ring focused pinhole collimator. For example, one collimator has a much smaller inner diameter, which gives a bigger magnification and resolution but could have a smaller field-of-view. Or one of the collimators has a much smaller field-of-view, which is better suited for imaging a smaller body part, e.g. the head or tumor vs. a full-body scan.

It is illustrated, as preferred, that each exchangeable collimator assembly not only includes one or more collimator components that make up a collimator but also includes a radiation shielding 18 that is then replaced together with the collimator.

In practical embodiments each exchangeable collimator assembly associated with the SPECT scanner device comprises not only that make up the collimator but also one or more shielding components that form an axial shield 18, 18′ in proximity an axial opening of the collimator. For example, as preferred, the assembly comprises two axial shields 18, 18″ as well as a more or less tubular collimator component. The shield 18, 18′ generally extends from said respective axial collimator opening outwards, e.g. conical, to a periphery of the shield 18, 18′ having greater cross-sectional dimensions than the respective axial opening. The housing 10 of the SPECT scanner may have, as shown in FIG. 3, a corresponding opening in a shield component 10a of the housing in which the axial shield of the assembly fits.

In some embodiments, the collimator in the SPECT scanner 5 may be rotatable by means of a collimator rotator device of the SPECT scanner device, which rotator device is not shown here. In such a case it is envisaged that the shielding 18, 18′ is held stationary, with the collimator being journalled relative to the shielding 18, 18′.

The SPECT scanner 5 further comprises a set of detectors 9 accommodated in housing 10. In embodiments, these detectors 9 are arranged stationary in the housing. In an alternative embodiment, the set of detectors 9 could be mounted to revolve over an angle about the imaging space 6.

In practical embodiments the set of detectors 9 forms a full ring around the SPECT scanning space 6. “Ring” should be understood in the sense that the detectors together form a detection surface that extends around the SPECT scanning space 6 in as much a closed-loop as possible. In practical embodiments a gap may be present between adjacent detectors 9 seen in circumferential direction around the imaging space.

In order for the control unit 20 to check whether the correct collimator has been placed in the SPECT scanner 5, an identification device 17 is provided on each collimator assembly, such as an RFID tag or a barcode, etc. The SPECT scanner is then provided with a corresponding reader, not shown here.

It is also possible that during SPECT scanning with one of the collimators 12, 13, the SPECT images as processed by the control unit 20 give rise to a replacement of the collimator for another one. This may e.g. be due to detected sources of radiation in the SPECT images. For example, it is well possible that the true region of interest 21 is not previously known, such as when looking for tumors or other marked tissue. In a preferred embodiment the system 1 is embodied such that it is possible to replace the collimator while the patient 2 is or remains in the SPECT scanning space 6, without interfering with the bed 2 or the position in the SPECT scanner. This is very helpful in efficient scanning with the thus adjusted SPECT scanner 6.

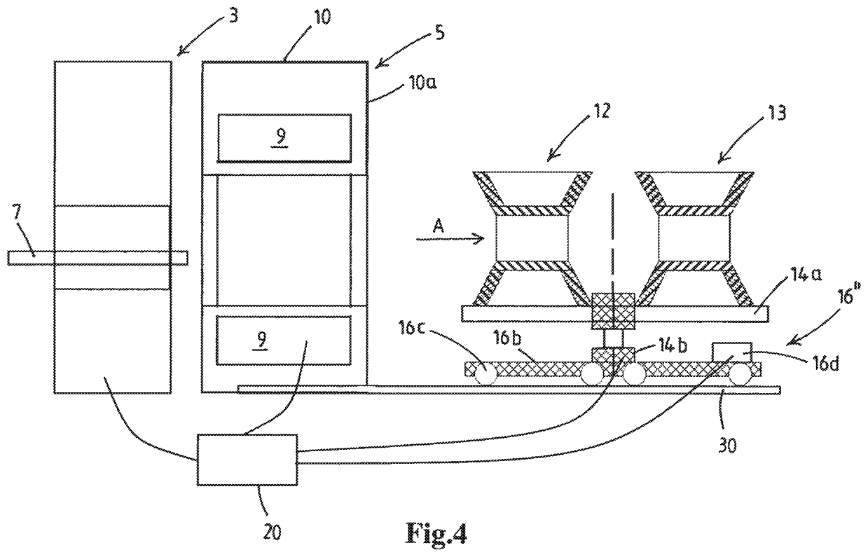

FIG. 4 illustrates an embodiment wherein the collimator support and conveyance system is configured to support at least two of the plurality of exchangeable collimator assemblies, for example the first collimator assembly 12 and the second collimator assembly 13, on a common carrier 14a, e.g. mounted on a cart 16″. As preferred, a carrier rotator 14b, e.g. motorized, is provided that is adapted to revolve the common carrier 14a about a vertical axis so as to allow aligning a selected one of said collimator assemblies 12, 13 on said common carrier 14a with the SPECT detector 5 for introduction of said selected collimator assembly into the SPECT scanner device. It is illustrated here that the common carrier 14a, is configured to support said first and second collimator assemblies 12, 13 at diametrically opposed support members of the common carrier 14a. The common carrier 14a is rotatable over 180° about the vertical axis in order to align a selected one of said collimator assemblies for introduction into the SPECT scanner device 5.

It is illustrated in FIG. 4 that the collimator support and conveyance system is an automated collimator support and conveyance system that is configured to automatically exchange a collimator assembly that is located in an operable position thereof where the collimator assembly surrounds the SPECT imaging space for another one of the plurality of exchangeable collimator assemblies, which other one collimator assembly is in a storage position thereof prior to said automated exchange. For example, the cart 16″ here is motorized with motor 16d to move in direction A and counter to said direction A for removal and introduction of an assembly 12, 13 from and into the SPECT scanner 5 at the side thereof facing away from the first scanner device 3.

It is illustrated in FIG. 4 that the cart 16″ is adapted to travel over a track, e.g. a floor bound track 30, e.g. the track extending in said direction A. The cart 16″ here is illustrated as being motorized to move over the track.

It will be appreciated that FIG. 4 illustrate a system that allows for a method for exchanging collimator assemblies in an imaging system, wherein the collimator support and conveyance system, here with cart 16″ and common carrier 14a, is used in removing one of the plurality of exchangeable collimator assemblies that is positioned in the SPECT scanner device 5 around the SPECT imaging space 6, said removal being done at the side of the housing 10 of the SPECT scanner device 5 that faces away from the first scanning device 3, and being done as preferred in direction A. The collimator support and conveyance system is then used in introduction of another one of the plurality of exchangeable collimator assemblies at the side of the housing 10 of the SPECT scanner device that faces away from the first scanning device 3, counter to the first direction A.

FIG. 4 illustrates that the control unit 20 that is linked to each of the first scanner device 3 and the SPECT scanner device 5, which control unit is configured to control each of the first scanner device 3 and the SPECT scanner device 5, is further configured to provide a selection instruction to select one collimator assembly from the plurality of exchangeable collimator assemblies 12, 13, based on a processing of the obtained scanning images of the first scanning device, e.g. by the control unit 20. This selection instruction is supplied to the automated collimator support and conveyance system, here to carrier rotator 14b and motor of cart 16″ in order to automatically position the selected collimator assembly around the SPECT imaging space.

1-19. (canceled)

20. An imaging system for obtaining images of a patient, the imaging system comprising:
a first scanning device, not being a SPECT scanner device, the first scanning device having a first imaging space and is configured to obtain scanning images of at least a part of the patient;
a second scanning device, being a SPECT scanner device, the second scanning device having a SPECT imaging space and being configured to obtain SPECT images of at least a part of the patient;
a patient carrier adapted to carry the patient;
a mover mechanism configured to move the patient carrier in a first direction allowing to introduce at least a part of the patient carried by the patient carrier into the first imaging space of the first scanning device,
wherein the SPECT scanner device is positioned behind the first scanning device when seen in the first direction,
wherein the mover mechanism is configured to move the patient carrier further in said first direction allowing to introduce at least a part of the patient carried by the patient carrier into the SPECT imaging space of the SPECT scanner device, and
wherein the SPECT scanner device comprises a housing accommodating a set of detectors in an arrangement around the SPECT imaging space;
a plurality of exchangeable collimator assemblies, wherein the SPECT scanner device is configured to receive one of said plurality of exchangeable collimator assemblies in a position around the SPECT imaging space, allowing to obtain SPECT images of at least a part of the patient using one or more of the detectors of the set of detectors,
wherein the plurality of exchangeable collimator assemblies comprises at least a first collimator assembly and a second collimator assembly, said first and second collimator assemblies having different imaging properties, and
wherein the SPECT scanner device is configured for removal and introduction of each one of said plurality of exchangeable collimator assemblies at a side of the housing thereof that faces away from the first scanning device; and
a collimator support and conveyance system configured to support and convey each collimator assembly of said plurality of exchangeable collimator assemblies in replacing one of the plurality of exchangeable collimator assemblies that is positioned in the SPECT scanner around the SPECT imaging space for another one of the plurality of exchangeable collimator assemblies.

21. The imaging system according to claim 20, wherein the imaging system is configured such that the imaging system allows for said replacing of one of the plurality of exchangeable collimator assemblies that is positioned in the SPECT scanner device around the SPECT imaging space for another one of the plurality of exchangeable collimator assemblies without affecting operability of the first scanner device.

22. The imaging system according to claim 20, wherein the imaging system is configured such that the imaging system allows for said replacing of one of the plurality of exchangeable collimator assemblies that is positioned in the SPECT scanner device around the SPECT imaging space for another one of the plurality of exchangeable collimator assemblies at least partially simultaneous with a scanning of the patient carried by the patient carrier with the first scanner device.

23. The imaging system according to claim 20, wherein each exchangeable collimator assembly associated with the SPECT scanner device comprises one or more collimator components that make up the collimator, the collimator defining the SPECT imaging space and having axial openings, and wherein each exchangeable collimator assembly associated with the SPECT scanner device further comprises one or more shielding components that each form an annular axial shield about one of the axial openings of the collimator.

24. The imaging system according to claim 20, wherein at least the one of the plurality of collimator assemblies is a full-ring collimator assembly configured to extend substantially completely around the SPECT imaging space during obtaining of the SPECT images.

25. The imaging system according to claim 20, wherein the SPECT scanner device comprises a collimator rotator device configured for rotating at least one of the plurality of collimator assemblies around the SPECT imaging space, at least over a predetermined angle.

26. The imaging system according to claim 20, wherein at least one of the plurality of exchangeable collimator assemblies is one of a focused pinhole collimator assembly, a cross-slit pinhole collimator assembly or a slit-slat collimator assembly.

27. The imaging system according to claim 20, wherein the collimator support and conveyance system is an automated collimator support and conveyance system configured to automatically exchange a collimator assembly located in an operable position thereof where the collimator assembly surrounds the SPECT imaging space for another one of the plurality of exchangeable collimator assemblies, the other one collimator assembly being in a storage position thereof prior to said automated exchange.

28. The imaging system according to claim 20, wherein the collimator support and conveyance system comprises a cart configured to support and convey at least one of said plurality of exchangeable collimator assemblies.

29. The imaging system according to claim 20, wherein the collimator support and conveyance system comprises a cart configured to support and convey at least one of said plurality of exchangeable collimator assemblies, said cart being adapted to travel over a floor, wherein a chassis of the cart is configured to be moved to under the region of the detectors of the SPECT scanner.

30. The imaging system according to claim 20, wherein the collimator support and conveyance system is configured to support said first collimator assembly and said second collimator assembly on a common carrier, the collimator support and conveyance system comprising a carrier rotator configured to revolve the common carrier about a vertical axis so as to allow aligning a selected one of said collimator assemblies on said common carrier with the SPECT detector for introduction of said selected collimator assembly into the SPECT scanner device.

31. The imaging system according to claim 20, further comprising a control unit linked to each of the first scanning device and the SPECT scanner device, the control unit being configured to control each of the first scanning device and the SPECT scanner device, wherein the control unit is further configured to provide control instructions to at least one of the SPECT scanner device and the mover mechanism based on a processing of obtained images of the first scanning device.

32. The imaging system according to claim 31, wherein said control unit is configured so that said processing of the obtained images of the first scanning device comprises determining a region of interest in the patient based on the obtained scanning images of the first scanning device, and wherein the control instructions comprise instructions to move the mover mechanism and to control the SPECT scanner such as to obtain one or more SPECT images of said determined region of interest.

33. The imaging system according to claim 31, wherein the control instructions comprise a selection instruction to select one collimator assembly from the plurality of exchangeable collimator assemblies, based on said processing of the obtained scanning images of the first scanning device.

34. The imaging system according to claim 33, wherein the collimator support and conveyance system is an automated collimator support and conveyance system configured to automatically exchange a collimator assembly located in an operable position thereof where the collimator assembly surrounds the SPECT imaging space for another one of the plurality of exchangeable collimator assemblies, the other one collimator assembly being in a storage position thereof prior to said automated exchange, and wherein the control unit is configured to supply the control instruction to the automated collimator support and conveyance system in order to automatically position the selected collimator assembly around the SPECT imaging space.

35. The imaging system according to claim 20, wherein each exchangeable collimator assembly associated with the SPECT scanner device comprises one or more collimator components that make up the collimator, the collimator defining the SPECT imaging space and having axial openings, and wherein each exchangeable collimator assembly associated with the SPECT scanner device further comprises one or more shielding components that each form an annular axial shield about one of the axial openings of the collimator, wherein the SPECT scanner device comprises a collimator rotator device configured for rotating at least one of the plurality of collimator assemblies around the SPECT imaging space, at least over a predetermined angle, wherein each annular axial shielding component is rotatably journalled relative to the collimator and being held stationary in the SPECT scanner device during a rotation of the collimator of the collimator assembly by the collimator rotator device.

36. The imaging system according to claim 20, wherein the first scanning device is embodied as a CT scanner or an MRI scanner.

37. A method for exchanging collimator assemblies in the imaging system of claim 20, wherein the collimator support and conveyance system is used in removing one of the plurality of exchangeable collimator assemblies positioned in the SPECT scanner device around the SPECT imaging space at the side of the housing of the SPECT scanner device that faces away from the first scanning device and is used in introduction of another one of the plurality of exchangeable collimator assemblies at the side of the housing of the SPECT scanner device that faces away from the first scanning device.

38. The method according to claim 37, wherein said removing of one of the plurality of exchangeable collimator assemblies and said introduction of another one of the plurality of exchangeable collimator assemblies at the side of the housing of the SPECT scanner device that faces away from the first scanning device is done whilst a patient carried by the patient carrier is in a position within the first imaging space.

39. The method according to claim 37, wherein the imaging system further comprises a control unit that is linked to each of the first scanner device and the SPECT scanner device, the control unit being configured to control each of the first scanner device and the SPECT scanner device, wherein the control unit is further configured to provide a selection instruction to select one collimator assembly from the plurality of exchangeable collimator assemblies, based on a processing of the obtained scanning images of the first scanning device.
